(19) United States
(12) Patent Application Publication
(10) Pub. No.: US 2015/0094344 A1
(43) Pub. Date: Apr. 2, 2015

(54) SHORT-ACTING DIHYDROPYRIDINES (CLEVIDIPINE) FOR USE IN REDUCING STROKE DAMAGE

(71) Applicant: Gregory Charles Williams, Mendham, NJ (US)

(72) Inventor: Gregory Charles Williams, Mendham, NJ (US)

(73) Assignee: THE MEDICINES COMPANY, Parsippany, NJ (US)

(21) Appl. No.: 14/009,212

(22) PCT Filed: Mar. 30, 2012
(86) PCT No.: PCT/US12/31449

§ 371 Date: Dec. 8, 2014

Related U.S. Application Data
(60) Provisional application No. 61/470,780, filed on Apr. 1, 2011.

(51) Int. Cl. A61K 31/4418

(52) U.S. Cl. CPC A61K 31/4418
USPC 514/356; 546/321

(57) ABSTRACT

The present invention relates to methods for lowering stroke damages and/or lowering blood pressure in a subject with a stroke in need thereof, comprising administering to the subject an effective amount of a pharmaceutical composition comprising a short acting dihydropyridine compound, preferably clevidipine or a pharmaceutically acceptable salt or ester thereof. Also provided are related medicaments, pharmaceutical compositions, and methods for preparing the medicaments.

### CROSS-REFERENCE TO RELATED APPLICATIONS

This application claims the benefit of U.S. Provisional Application No. 61/470,780, filed on Apr. 1, 2011, the contents of which are incorporated by reference herein, in their entireties and for all purposes.

### BACKGROUND OF THE INVENTION

A stroke is an interruption of blood supply to the brain because a blood vessel is blocked or bursts open. When blood flow is stopped, the brain cannot get oxygen and nutrients, and brain cells can die causing permanent damage. The effects of a stroke depend on which part of the brain is injured and how severely it is affected. A very severe stroke can cause sudden death.

After stroke, blood pressure is often elevated within hours. For every 10-mm Hg increase over 180 mm Hg, the risk of neurological deterioration increases by 40% and the risk of poor outcome increases by 23%. Adams et al., Stroke 38:1655-1711, 1670 (2007). Lowering blood pressure can reduce damage caused by the stroke, for example, formation of brain edema, hemorrhagic transformation of the infarction, vascular damage, and early recurrent stroke. Aggressively lowering blood pressure among stroke patients however may lead to neurological worsening by reducing perfusion pressure to ischemic areas of the brain. Adams et al., Stroke 38:1655-1711, 1670 (2007). Thus, blood pressure management in stroke patients poses a dilemma since severe elevations of blood pressure may place inordinate strain on the heart and other vital organs yet lowering this elevated blood pressure may reduce the blood flow to areas of the brain that are already receiving lesser amounts of blood. Accordingly, the American Heart Association recommends treating arterial hypertension in acute ischemic stroke patients with anti-hypertensive agents having “the potential for a rapid reversal if the drop in blood pressure leads to neurological worsening” with the goal to avoid overtreating patients with a stroke. Adams et al., Stroke 38:1655-1711, 1670-1671 (2007).

Therefore, there remains a need for an anti-hypertensive drug that provides an optimal balance of efficacy, precision (titratability), and safety in stroke patients, especially acutely unstable patients.

### SUMMARY OF THE INVENTION

The present invention relates to the use of a short active dihydropyridine compound for reducing stroke damage and/or lowering blood pressure in a subject with a stroke, and pharmaceutical compositions or medicaments comprising the short active dihydropyridine compound.

A method for reducing stroke damage in a subject with a stroke in need thereof is provided. Also provided is a method for lowering blood pressure and reducing stroke damage in a subject with a stroke in need thereof. These methods comprise administering to the subject an effective amount of a pharmaceutical composition comprising a short acting dihydropyridine compound. Discontinuation of administering the short acting dihydropyridine compound may allow return of the blood pressure to a pre-treatment level in the subject within 30 minutes. The short acting dihydropyridine compound may have a short half life in plasma of less than 30 minutes, and is preferably clevidipine or a pharmaceutically acceptable salt or ester thereof.

The method may further comprise titrating the dose of the pharmaceutical composition from an initial dose to a maintenance dose by multiple dosage adjustments, whereby a desired blood pressure is achieved in the subject. The time interval between dosage adjustments may be 5-10 minutes. Each dosage adjustment is preferably less than doubling.

The subject is a mammal, preferably a human. The subject may have severe hypertension.

The stroke may be an ischemic stroke or a hemorrhragic stroke. The ischemic stroke may be a transient ischemic attack. The hemorrhagic stroke may be due to intracranial hemorrhage. In particular, the intracranial hemorrhage may be intracerebral hemorrhage (ICH).

The stroke damage may be neurological worsening, brain injury or death. It may be permanent.

In a method according to the present invention, the pharmaceutical composition may comprise 0.001-20 mg/ml, preferably 0.5 mg/ml, clevidipine or a pharmaceutically acceptable salt or ester thereof The pharmaceutical composition may further comprise a pharmaceutically acceptable carrier or diluent. Preferably, the pharmaceutical composition has a pH of 6.0-8.8.

The pharmaceutical composition may be an emulsion. The emulsion may comprise a lipid at 2-30% mg/ml and an emulsifier at 0.2-2 mg/ml. The pharmaceutical composition may further comprise one or more agents selected from the group consisting of an antimicrobial agent, a tonicity modifier, an antioxidant, and a co-emulsifier.

For each of the methods described herein, a medicament comprising an effective amount of a short acting dihydropyridine compound is provided. The medicament is useful for reducing stroke damage and/or lowering blood pressure in a subject. The short acting dihydropyridine compound preferably has a half life in plasma of less than 30 minutes. More preferably, the short acting dihydropyridine compound is clevidipine or a pharmaceutically acceptable salt or ester thereof.

A medicament according to the present invention may comprise about 0.001-20 mg/ml, preferably 0.5 mg/ml, clevidipine or a pharmaceutically acceptable salt or ester thereof (e.g., clevidipine butyrate). The medicament may further comprise a pharmaceutically acceptable carrier or diluent. The medicament may be an emulsion, comprising a lipid at 2-30% mg/ml and an emulsifier at 0.2-2 mg/ml. The medicament may further comprise an antimicrobial agent, a tonicity modifier, an antioxidant, and/or a co-emulsifier. The pH of the medicament is in the range of 6.0-8.8.

A method for preparing a medicament useful for reducing stroke damage and/or lowering blood pressure in a subject with a stroke is provided. The method may comprise admixing a short acting dihydropyridine compound with a pharmaceutically acceptable carrier or diluent. The method may also comprise admixing a short acting dihydropyridine compound with a lipid, an emulsifier, and water. The method may further comprise adding an antimicrobial agent, a tonicity modifier, an antioxidant, and/or a co-emulsifier. The method may also further comprise adjusting the pH of the admixture to 6.0-8.8; and/or placing the medicament in a sterile pre-filled syringe.

### DETAILED DESCRIPTION OF THE INVENTION

The present invention is based on the discovery that clevidipine, a short acting dihydropyridine compound, is effective in reducing stroke damage and/or lowering blood pressure in a patient with a stroke. In particular, clevidipine has “the potential for a rapid reversal if the drop in blood pressure leads to neurological worsening” as recommended by the American Stroke Association for an anti-hypertensive agent for patients with a stroke. Clevidipine provides the optimal balance of efficacy, precision (titratability), and safety.

Clevidipine is a dihydropyridine L-type calcium channel blocker. Having a very short half-life (about 1 minute), clevidipine exhibits rapid onset of activity (2 to 4 minutes) and rapid offset of activity (full offset of activity in 5 to 15 minutes). The chemical structure of clevidipine is shown in Formula I.

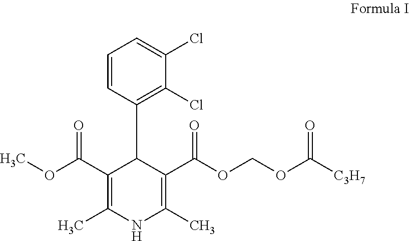

The term “clevidipine” as used herein encompasses the compound of Formula I, as well as tautomeric, enantiomeric and diastereomeric forms thereof, and racemic mixtures thereof, other chemically active forms thereof, and pharmaceutically acceptable salts, esters, isomers, stereo isomers, crystalline and amorphous forms of these compounds. One particular example is clevidipine butyrate. These alternative forms and salts, processes for their production, and pharmaceutical compositions comprising them, are well known in the art and set forth in U.S. Pat. Nos. 5,856,346, 5,739,152, and 6,350,877, as well as International Patent Application Nos. PCT/US09/004399 and PCT/US09/52127.

The present invention provides various methods, including a method for reducing stroke damage in a subject with a stroke in need thereof, and a method for lowering blood pressure and reducing stroke damage in a subject with a stroke in need thereof. These methods comprise administering to the subject an effective amount of a pharmaceutical composition comprising a short acting dihydropyridine compound. An example of the short acting dihydropyridine compound is clevidipine. Other short acting dihydropyridine compounds may include compounds corresponding to formula I as set forth in U.S. Pat. No. 5,739,152, and formula I as set forth in U.S. Pat. No. 5,856,346, and pharmaceutical acceptable salts, esters, isomers, stereo isomers, crystalline and amorphous forms thereof.

The short acting dihydropyridine compound may have a half life in plasma of less than about 30, 15, 10, 5, or 2 minutes, preferably less than about 10 minutes, more preferably less than about 5 minutes, most preferably less than about 2 minutes. The short acting dihydropyridine compound has a rapid onset of activity as well as a rapid offset of activity. A short acting drug reaches steady plasma drug concentration quickly (e.g., within less than about one hour, 45 minutes, 30 minutes, 15 minutes, 10 minutes, or 5 minutes after starting drug administration), and gets cleared quickly (e.g., within about five hours, three hours, one hour, 30 minutes, 15 minutes, 10 minutes, 5 minutes, 3 minutes, 2 minutes, 1 minute, or 30 seconds after ending drug administration). The full offset of activity may be achieved within about one hour, 45 minutes, 30 minutes, 15 minutes, 10 minutes, 5 minutes, 3 minutes, 2 minutes, or 1 minute, preferably within 5-15 minutes. The short acting dihydropyridine compound is preferably clevidipine or a pharmaceutically acceptable salt or ester thereof.

A method according the present invention may comprise titrating the dose of the pharmaceutical composition from an initial dose to a maintenance dose by multiple dosage adjustments such that a desired blood pressure is achieved in the subject. The initial dose may be about 0.1-20 mg/hour, preferably about 1-2 mg/hour, of clevidipine or a pharmaceutically acceptable salt or ester thereof. The maintenance dose may be about 0.1-50, 1-32, 1-16, or 4-6 mg/hour of clevidipine or a pharmaceutically acceptable salt or ester thereof. The time interval between dosage adjustments is about 1-30 minutes, preferably 2-20 minutes, more preferably about 5-10 minutes. Each dosage adjustment is preferably less than doubling.

The stroke may be ischemic or hemorrhagic. An ischemic stroke may be due to thrombosis or embolism, which may be of cardiac origin. An ischemic stroke may be a transient ischemic attack. It may also be total anterior circulation infarct (TACI), partial anterior circulation infarct (PACI), lacunar infarct (LACI), or posterior circulation infarct (POCI). A hemorrhagic stroke may be due to intracerebral hemorrhage, which may be intra-axial or extra-axial. The intra-axial hemorrhage may be intraparentchymal or intraventricular hemorrhage. The extra-axial hemorrhage may be epidural, subdural or subarachnoid hemorrhage. In particular, the intracranial hemorrhage may be intracerebral hemorrhage (ICH). Further, the stroke may be selected from the group consisting of right-hemisphere stroke, left-hemisphere stroke, cerebella stroke, and brain stem stroke.

In a method according to the present invention, the stroke damage may be neurological worsening (or neurological deterioration), which may take place within about 3 months, 1 month, 1 week, 2 days or 1 day after the stroke. The stroke damage may be brain injury caused by, for example, a hematoma. The hematoma may be subgaleal hematoma, cephalohematoma, epidural hematoma, subdural hematoma, subarachnoid hematoma, or othematoma. Reducing stroke damage may be, for example, reducing the expansion (or size) of a brain hematoma. The stroke damage may also be brain edema, cerebral infarction, hemorrhagic transformation of cerebral infarction, vascular damage, or a recurrent stroke. The recurrent stroke may occur within about 6 months, 3 months, 1 month, 2 weeks or 1 week after the stroke. Further, the stroke damage may be hypoxia, increased body temperature, hypoglycemia, hyperglycemia or death, which may occur within about 3 months, 1 month, 1 week, 2 days or 1 day after the stroke. In some embodiments, the stroke damage is permanent.

Discontinuation of administering the short acting dihydropyridine compound may allow return of the blood pressure to a pre-treatment level in the subject, for example, within about 30, 20, 15, 10, 5 or 3 minutes, preferably about 15 minutes, more preferably about 10 minutes, most preferably about 5 minutes. The one minute half-life of clevidipine results in a rapid offset of action with the return of blood pressure to pre-treatment levels within about 5-15 minutes of the discontinuation of clevidipine.

The subject is a mammal, for example, a mouse, rat, dog, pig, or human, preferably a human. The subject may be male or female. The subject may be at least 50, 55, 60 or 65 years old, preferably at least 55 years old. The subject may have severe hypertension, and/or arterial hypertension. In the subject, the systolic blood pressure may be at least about 160, 180, 185, 220, or 230 mm Hg, and/or the diastolic blood pressure may be at least about 105, 110, 120, or 140 mm Hg.

The subject may have suffered from hypertensive encepthalopathy, aortic dissection, acute renal failure, acute pulmonary edema, or acute myocardial infarction. The subject may also have suffered from atrial fibrillation, diabetes, a family history of stroke, a previous stroke, a previous transient ischemic attack, a heart disease, high cholesterol, or sickle cell anemia.

The subject may have suffered from thrombosis. The thrombosis may be a large vessel disease or a small vessel disease. The large vessel disease may be atherosclerosis, vasoconstriction, aortic, carotid or vertebral artery dissection, an inflammatory disease of a blood vessel wall, noninflammatory vasculopathy, Moyamoya disease, or fibromuscular dysplasia. The inflammatory disease of a blood vessel wall may be selected from the group consisting of Takayasu arteritis, giant cell arteritis, and vasculitis. The small vessel disease may be lipohyalinosis, fibrinoid degeneration, or microatheroma.

The subject may have received an anti-hypertensive drug or an anticoagulation drug. The anti-hypertensive drug may be, for example, thiazide diuretics, angiotensin-converting enzyme (ACE) inhibitors, calcium channel blockers, beta blockers, or angiotensin II receptor antagonists. The anticoagulation drug may be warfarin, aspirin, or antiplatelet drugs.

Clevidipine is an ideal parenteral anti-hypertensive medication as it provides an optimal balance of efficacy (the ability to rapidly reduce blood pressure to target levels), safety (the ability to avoid overshoot hypotension, and absence of toxicity and side-effects), and precision (the ability to hit and maintain blood pressure target levels while avoiding overshoot, and the speed with which titration can be accomplished). Additionally, in patients with pre-existing or inter-current hepatic or renal dysfunction, agents that are metabolized renally or hepatically are unsuitable.

Because of its rapid onset and offset, clevidipine can be titrated in a manner allowing rapid upward and downward adjustments in dose as clinical circumstances dictate, and substantially reducing the risk of overshoot hypotension, which is especially important in hemodynamically unstable patients. Clevidipine is rapidly metabolized via blood and tissue esterases, and does not accumulate in tissues. It can therefore be safely administered to hepatically and renally compromised patients.

The term “an effective amount” refers to an amount of a pharmaceutical composition comprising a short acting dihydropyridine compound (e.g., clevidipine) required to achieve a stated goal (e.g., reducing stroke damage and/or lowering blood pressure). The effective amount of the pharmaceutical compositions comprising a short acting dihydropyridine compound (e.g., clevidipine) may vary depending upon the stated goals, the physical characteristics of the subject, the nature and severity of the stroke damage and/or hypertension, existence of related or unrelated medical conditions, the nature of the short acting dihydropyridine compound, the composition comprising the short acting dihydropyridine compound (e.g., clevidipine), the means of administering the drug to the subject, and the administration route. A specific dose for a given subject may generally be set by the judgment of a physician. The pharmaceutical composition may be administered to the subject in one or multiple doses.

The pharmaceutical composition may comprise about 0.001-20, 0.005-1, 0.01-1, or 0.05-0.5 mg/ml, preferably 0.5 mg/ml, clevidipine or a pharmaceutically acceptable salt or ester thereof. The pharmaceutical composition may further comprise a pharmaceutically acceptable carrier or diluent. Carriers, diluents and excipients suitable in the pharmaceutical composition are well known in the art. Suitable pharmaceutical compositions include the formulations (e.g., solutions and emulsions) described in U.S. Pat. Nos. 5,856,346, 5,739,152, and 6,350,877, as well as International Patent Application Nos. PCT/US09/004399 and PCT/US09/52127.

The pharmaceutical composition may have a pH of about 5.6-10.0, preferably 6.0-8.8, more preferably 6.5-8.0. For example, the pH may be about 6.2, 6.5, 6.75, 7.0, or 7.5.

The pharmaceutical composition may be an emulsion, freeze dried material from the emulsion, or a concentrate for reconstitution (self-emulsifying system). Preferably, the pharmaceutical composition is an emulsion. The emulsion may comprise a short acting dihydropyridine compound, a lipid, an emulsifier, and water or a buffer. The lipid may be present at about 2-30% mg/ml, and selected from the group consisting of soybean oil, safflower seed oil, olive oil, cottonseed oil, sunflower oil, sesame oil, peanut oil, corn oil, medium chain triglycerides, triacetin, propylene glycol diesters, monoglycerides, and a mixture of two or more thereof. The emulsifier may be present at about 0.2-2 mg/ml, and be selected from the group consisting of egg yolk phospholipids, soybean phospholipids, synthetic phosphatidyl cholines, purified phosphatidyl cholines and hydrogenated phosphatidyl choline, and mixtures of two or more thereof.

The pharmaceutical composition may also comprise an antimicrobial agent, a tonicity modifier, an antioxidant, and/or a co-emulsifier. The antimicrobial agent may be present at about 0.01-1 mg/ml, and selected from the group consisting of benzyl alcohol, EDTA, sodium ascorbate, citric acid, and mixtures, derivatives, and salts thereof. The tonicity modifier may be present at about 2-3 mg/ml. The antioxidant may be present at about 0.01-1 mg/ml, and selected from the group consisting of sodium ascorbate, sodium citrate, cysteine hydrochloride, sodium bisulfate, sodium metabisulfite, sodium sulfite ascorbyl palmitate, butylated hydroxyanisole (BHA), butylated hydroxytoluene (BHT), propyl gallate, tocopherol, and a pharmaceutically acceptable salt thereof. The co-emulsifier is present at about 0.01-2 mg/ml, and may be selected from the group consisting of glycerol (or glycerin), poloxamers, Cremophor™, poloxamines, polyoxyethylene stearates, polyoxyethylene sorbitan fatty acid esters, sorbitan fatty acid esters, polysorbates, tocopherol PEG succinate, cholic acid, deoxycholic acid, oleic acid, and pharmaceutically acceptable salts thereof.

The pharmaceutical compositions of the present invention may be formulated, for example, for oral, sublingual, intranasal, intraocular, rectal, transdermal, mucosal, topical or parenteral administration. Parenteral administration may include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any device suitable for parenteral injection or infusion of drug formulations may be used for such administration. For example, the pharmaceutical composition may be contained in a sterile pre-filled syringe.

According to the present invention, the pharmaceutical compositions are preferably administered to the subject in a parental dosage form, more preferably in an intravenous dosage form. The intravenous dosage form may be a bolus intravenous dosage form or a continuous intravenous infusion dosage form, preferably a continuous intravenous infusion dosage form.

When administered as a continuous intravenous infusion dosage form, the pharmaceutical composition may be administered to the subject at about 0.1-100, 0.1-50, 0.1-25, 0.1-10, 0.1-7.5, 0.1-5, 0.1-2.5, 0.1-2, 0.1-1, or 0.1-0.5 μg clevidipine, or a pharmaceutically acceptable salt or ester thereof, per kg body weight per minute (e.g., about 0.1, 0.5, 1, 2, 5, 7.5, 10, 15, 20, 25, or 30 μg/kg/min), preferably about 1-10 μg/kg/min. The pharmaceutical composition may be administered continuously for a period of at least about 0.1, 0.2, 0.5, 1, 1.5, 2, 2.5, 3, 3.5, or 4 hours.

In some embodiments, medicaments comprising an effective amount of a short acting dihydropyridine compound are provided. The medicaments are useful for reducing stroke damage and/or lowering blood pressure. The short acting dihydropyridine compound preferably has a short half life in plasma (e.g., less than about 30, 15, 10, 5, or 2 minutes). The short acting dihydropyridine compound is preferably clevidipine or a pharmaceutically acceptable salt or ester thereof.

The medicament may comprise about 0.001-20, 0.005-1, 0.01-1, or 0.05-0.5 mg/ml, preferably about 0.5 mg/ml, clevidipine or a pharmaceutically acceptable salt or ester thereof. The medicament may further comprise a pharmaceutically acceptable carrier or diluent.

The medicament may be an emulsion, comprising a lipid and an emulsifier. The lipid may be present at about 2-30% mg/ml. The emulsifier may be present at about 0.2-2 mg/ml.

The medicament may further comprise one or more agents selected from the group consisting of an antimicrobial agent, a tonicity modifier, an antioxidant, and a co-emulsifier, which may be present at about 0.01-1 mg/ml, 2-3 mg/ml, 0.01-1 mg/ml, and 0.01-2 mg/ml, respectively.

The medicament may have a pH of about 5.6-10.0, preferably about 6.0-8.8, more preferably about 6.5-8.0. For example, the pH may be about 6.2, 6.5, 6.75, 7.0, or 7.5. The medicament may be contained in a sterile pre-filled syringe.

In some other embodiments, methods for preparing the medicaments according to the present invention are provided. The preparation methods may comprise a short acting dihydropyridine compound with a pharmaceutically acceptable carrier or diluent. The preparation methods may also comprise combining a short acting dihydropyridine compound with a lipid, an emulsifier, and water. The short acting dihydropyridine compound may have a short half life in plasma (e.g., less than about 30, 15, 10, 5, or 2 minutes). Preferably, the short acting dihydropyridine compound is clevidipine or a pharmaceutically acceptable salt or ester thereof. The methods may further comprise adding one or more agents selected from the group consisting of an antimicrobial agent, a tonicity modifier, an antioxidant, and a co-emulsifier; adjusting the pH of the admixture to about 6.0-8.8; and/or placing the medicament in a sterile pre-filled syringe.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A composition comprising an effective amount of a short acting dihydropyridine compound for reducing stroke damage in a subject with a stroke.

2. The medicament of claim 1, wherein the composition is effective for lowering blood pressure in the subject.

3. The medicament of claim 1, wherein the short acting dihydropyridine compound has a half life in plasma of less than 30 minutes.

4. The medicament of claim 1, wherein the short acting dihydropyridine compound is clevidipine or a pharmaceutically acceptable salt or ester thereof.

5. The medicament of claim 4, wherein the medicament comprises 0.001-20 mg/ml clevidipine or a pharmaceutically acceptable salt or ester thereof.

6. The medicament of claim 1, wherein the medicament is an emulsion.

7. The medicament of claim 6, wherein the emulsion comprises a lipid at 2-30% mg/ml.

8. The medicament of claim 6, wherein the emulsion comprises an emulsifier at 0.2-2 mg/ml.

9. The medicament of claim 1, further comprising one or more agents selected from the group consisting of an antimicrobial agent, a tonicity modifier, an antioxidant, and a co-emulsifier.

10. The medicament of claim 1, wherein the medicament has a pH of 6.0-8.8.

11. The medicament of claim 1, wherein the medicament is contained in a sterile pre-filled syringe.

12. A method for reducing stroke damage in a subject with a stroke, comprising administering to the subject an effective amount of a composition comprising a short acting dihydropyridine compound.

13. The method of claim 12, wherein the composition further comprises a pharmaceutically acceptable carrier or diluet.

14. A method for lowering blood pressure and reducing stroke damage in a subject with a stroke, comprising administering to the subject an effective amount of a composition comprising a short acting dihydropyridine compound.

15. The method of claim 14, wherein the composition further comprises a pharmaceutically acceptable carrier or diluent.

16. The method of claim 12, wherein the short acting dihydropyridine compound has a half life in plasma of less than 30 minutes.

17. The method of claim 12, wherein the short acting dihydropyridine compound is clevidipine or a pharmaceutically acceptable salt or ester thereof.

18. The method of claim 12, further comprising adding one or more agents selected from the group consisting of an antimicrobial agent, a tonicity modifier, an antioxidant, and a co-emulsifier.

19. The method of claim 12, further comprising adjusting the pH of the admixture to 6.0-8.8.

20. The method of claim 12, further comprising placing the medicament in a sterile pre-filled syringe.
